(19) United States
(12) Patent Application Publication
(10) Pub. No.: US 2014/0309489 A1
(43) Pub. Date: Oct. 16, 2014

(54) G-SPOT LATEX CONDOM

(71) Applicant: AILIN FERNANDEZ, MIAMI, FL (US)

(72) Inventor: AILIN FERNANDEZ, MIAMI, FL (US)

(21) Appl. No.: 13/862,368

(22) Filed: Apr. 12, 2013

(51) Int. Cl. A61F 6/04; A61H 19/00

(52) U.S. Cl. CPC A61F 6/04; A61H 19/44; A61H 19/32; A61F 2006/048
USPC 600/38

(57) ABSTRACT

A g-spot condom that stimulates the g-spot of a woman. The g-spot condom comprises of a sheath, the sheath defines: a head section, the head section has a tip section and a bottom section, and an inner and outer side; a body section, the body section defines a first a and a second side, and an inner and an outer side, the first side of the body section is adjacent to the bottom section of the head section; a securing section, the securing section is adjacent to the second side of the body section; and at least one pearl shaped bulb that extends outward from the sheath.

### CROSS REFERENCE TO RELATED APPLICATIONS

This application claims the benefit of the filing dates of U.S. Provisional Patent Application No. 61/737,286, filed on Dec. 14, 2012, the disclosure of which is incorporated herein by reference.

### BACKGROUND

The present invention is directed to a condom that is fabricated to stimulate the g-spot of a woman while simultaneously providing extra sensation to the man wearing the condom.

The woman's g-spot has been described as being located one to three inches (2.5 to 7.6 cm) up the front (anterior) vaginal wall between the vaginal opening and the urethra and is believed to be a sensitive area that some women report is their erogenous zone. Some women state that when the zone is stimulated they become more aroused and that they experience greater orgasms.

A condom is defined as being a thin rubber or latex sheath worn on a man's penis during sexual intercourse as a contraceptive and/or as protection against infection. Most condoms are presently made of latex. Some condoms are designed to give the man greater stimulation. Most condoms are not designed to heightening the stimulation of the woman.

Condoms made to heighten a woman's stimulation are usually ribbed condoms. They are meant to provide ridges that stimulate the vagina of the woman. Ribbed condoms are not designed to stimulate a woman's g-spot.

The present invention is a condom that is specifically designed to stimulate the g-spot of the woman while simultaneously stimulating the penis of the man.

### SUMMARY

The present invention is directed towards a condom that the g-spot of a woman while simultaneously stimulating the penis of the man.

The g-spot condom defines a head section, the head section has a tip section and a bottom section, and an inner and outer side; a body section, the body section defines a first and a second side, and an inner and an outer side, the first side of the body section is adjacent to the bottom section of the head section; and a securing section, the securing section is adjacent to the second side of the body section. The head section of the condom defines at least three pearl shaped bulbs that flow outward from the bottom section of the head section that are equidistant from each other. The aforesaid is the simplest embodiment of the present invention.

In another embodiment of the present invention, inner side of the condoms head and body section shall define a plurality of small bulbs. The small bulbs are displaced throughout the head and body inner sections of the condom.

In a further embodiment of the present invention, the tip section of the head section shall define a set of rabbit ears.

In still a further embodiment of the present invention, a ring shall be defined at the juncture between the head section and body section, the ring shall be a ridge that shall be at least 2 centimeters in width.

In yet another embodiment of the present invention, the ring section shall define bulbs that extrude outward from the condom.

In the preferred embodiments of the present invention, the condoms are fabricated of latex and are made using a mold. The condoms can be multi-colored. The colors can be deep colors and they may even be fluorescent.

The pearl shaped bulbs that flow outward from the bottom section of the head section of the condom are designed to stimulate the g-spot of the woman. The reason that the pearl shaped bulbs are equidistant from each other is to ensure that the user of the condom maximizes the chances of placing the pearl shaped bulbs in the correct location when the condom is worn.

The plurality of small bulbs in the inside of the condom are designed to enhance the sensitivity felt by the user of the condom.

The rabbit ears of one of the embodiments of the present invention are designed to further stimulate the vagina of a woman.

The ring section of the present invention is designed to provide further stimulation to the woman's vagina, more so when the ring section defines the bulbs that extrude outward.

An object of the present invention is to provide a condom that will stimulate the g-spot of a woman.

Another object of the present invention is to provide a condom that will provide greater stimulation or sensitivity to the male user of the condom.

A further object of the present invention is to provide a deep colored condom that can be enjoyed by the users of the condom.

### DRAWINGS

These and other features, aspects, and advantages of the present invention will become better understood with regard to the previous description and drawings where:

FIG. 1 is a perspective view of one of the embodiments of the present invention; and

FIG. 2 is a perspective view of another embodiment of the present invention.

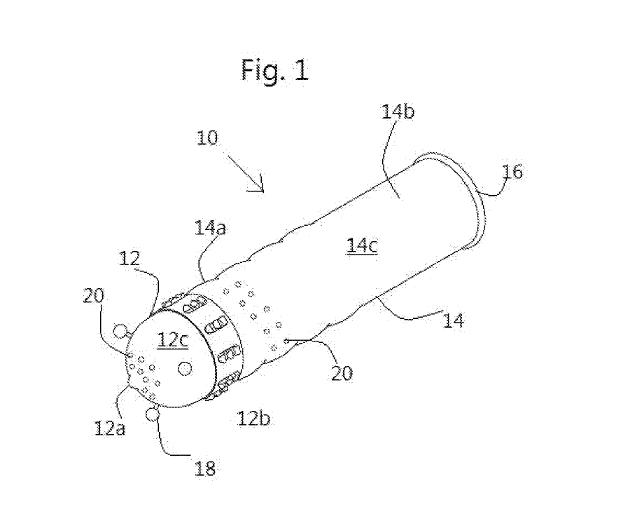

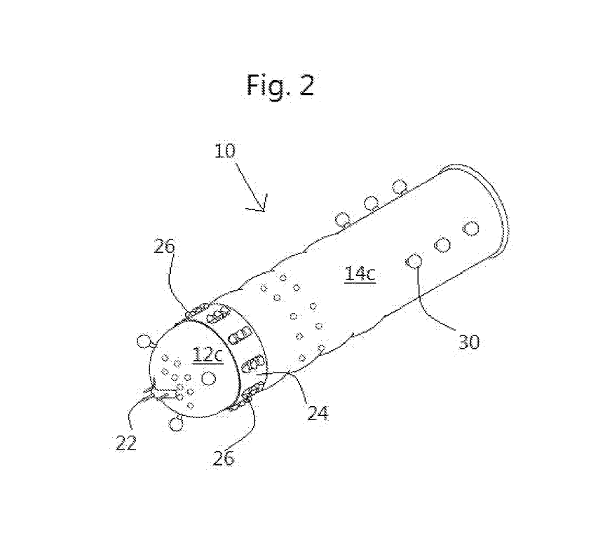

As seen in FIGS. 1 and 2, the g-spot condom of the present invention comprises of a sheath 10. The sheath 10 defines a head section 12. The head section 12 has a tip section 12a and a bottom section 12b, and an inner (not shown in figures) and outer side 12c. The sheath 10 further defines a body section 14. The body section 14 defines a first 14a and a second side 14b, and an inner (not shown in figures) and an outer side 14c, the first side of the body section 14a is adjacent to the bottom section of the head section 12b. And, a securing section 16. The securing section 16 is adjacent to the second side of the body section 14b. The sheath 10 defines at least one pearl shaped bulb 18, 30 that extends outward from the sheath 10.

In another embodiment of the present invention, the inner side of the sheath's head and body section shall define a plurality of small bulbs 20. The small bulbs 20 are displaced throughout the head 12 and body inner sections 14 of the sheath 10.

In a further embodiment of the present invention, the tip section 12a of the head section 12 shall define a plurality of extensions 22 extending outward from the tip section 12a.

In still a further embodiment of the present invention, a ring 24 shall be defined at the juncture between the head section and body section, the ring 24 shall be a ridge that shall be at least 2 centimeters in width.

In yet another embodiment of the present invention, the ring section 24 shall define ring bulbs 26 that extend outward from the sheath 10.

In another embodiment of the present invention, each pearl shaped bulb 18 shall be displaced on the head section 12 of the sheath 10.

In another embodiment of the present invention, each pearl shaped bulb 30 shall be displaced on the body section 14 of the sheath 10. In a preferred embodiment, each pearl shaped bulb 30 shall be displaced a distance between 2 and 8 cm from the securing section 16.

In the preferred embodiments of the present invention, the condoms are fabricated of latex and are made using a mold. The condoms can be multi-colored. The colors can be deep colors and they may even be fluorescent.

An advantage of the present invention is that it provides a condom that stimulates the g-spot of a woman.

Another advantage of the present invention is that it provides a condom that provides greater stimulation or sensitivity to the male user of the condom.

A further advantage of the present invention is that it provides a deep colored condom that can be enjoyed by users of the condom.

Although the present invention has been described in considerable detail in reference to preferred versions, other versions are possible. Therefore, the spirit and scope of the appended claims should not be limited to the description of the preferred versions contained herein.

What is claimed is:

1. A g-spot condom, the g-spot condom comprises of a sheath, the sheath defines:
a head section, the head section has a tip section and a bottom section, and an inner and outer side;
a body section, the body section defines a first a and a second side, and an inner and an outer side, the first side of the body section is adjacent to the bottom section of the head section;
a securing section, the securing section is adjacent to the second side of the body section; and
at least one pearl shaped bulb that extends outward from the sheath.

2. The g-spot condom of claim 1, further defining a plurality of small bulbs throughout the head and body inner sections of the sheath.

3. The g-spot condom of claim 2, further defining a plurality of extensions extending outward from the tip section of the head section.

4. The g-spot condom of claim 3, further defining a ring, the ring is at a juncture between the head section and body section, the ring is a ridge.

5. The g-spot condom of claim 4, wherein the ring section defines a plurality of ring bulbs that extends outward from the sheath.

6. The g-spot condom of claim 5, each pearl shaped bulb is displaced on the body section of the sheath.

7. The g-spot condom of claim 4, wherein each pearl shaped bulb is displaced a distance between 2 and 8 cm from the securing section.

9. The g-spot condom of claim 8, wherein the pearl shaped bulbs are equidistant from each other.

10. The g-spot condom of claim 5, wherein each pearl shaped bulb is displaced on the head section of the sheath.

11. The g-spot condom of claim 10, wherein the pearl shaped bulbs are equidistant from each other.

12. The g-spot condom of claim 2, each pearl shaped bulb is displaced on the body section of the sheath.

13. The g-spot condom of claim 12, wherein each pearl shaped bulb is displaced a distance between 2 and 8 cm from the securing section.

14. The g-spot condom of claim 13, wherein the pearl shaped bulbs are equidistant from each other.

15. The g-spot condom of claim 14, further defining a plurality of small bulbs throughout the head and body inner sections of the sheath.

16. The g-spot condom of claim 15, further defining a plurality of extensions extending outward from the tip section of the head section.

17. The g-spot condom of claim 16, further defining a ring, the ring is at a juncture between the head section and body section, the ring is a ridge.

18. The g-spot condom of claim 17, wherein the ring section defines a plurality of ring bulbs that extends outward from the sheath.

19. The g-spot condom of claim 2, further defining a ring, the ring is at a juncture between the head section and body section, the ring is a ridge.

20. The g-spot condom of claim 19, wherein the ring section defines a plurality of ring bulbs that extends outward from the sheath.
